(19) United States
(12) Patent Application Publication
(10) Pub. No.: US 2006/0045923 A1
(43) Pub. Date: Mar. 2, 2006

(54) Composition containing tinospora cordifolia and process for obtaining same

(76) Inventors: Lal Hingorani, Khan West (IN); Vijay Thawani, Clarke Town (IN)

Correspondence Address: AMIN LAW, LLC, 217 N. JEFFERSON ST., STE. 500, CHICAGO, IL, 60661, US

(21) Appl. No.: 11/219,117

(22) Filed: Sep. 2, 2005

Related U.S. Application Data
(60) Provisional application No. 60/606,749, filed on Sep. 2, 2004.

(51) Int. Cl. A61K 36/185

(52) U.S. Cl. 424/725

(57) ABSTRACT

The present invention relates to a composition for use in a nutritional product, dietary supplement, food or pharmaceutical composition wherein such composition includes the plant Tinospora cordifolia or Tinospora cordifolia extract and is used to treat allergies, including but not limited to seasonal, perennial, or sporadic allergic rhinitis. The present invention further relates to an extraction process for obtaining such composition.

### CROSS REFERENCE TO RELATED APPLICATIONS

This application claims the benefit of U.S. provisional application No. 60/606,749, filed on Sep. 2, 2004.

### FIELD OF INVENTION

The present invention relates to a composition for use in a nutritional product, dietary supplement, food or pharmaceutical composition wherein such composition includes the plant Tinospora cordifolia or Tinospora cordifolia extract and is used to treat allergies, including but not limited to seasonal, perennial, or sporadic allergic rhinitis. The present invention further relates to an extraction process for obtaining such composition.

### BACKGROUND OF THE INVENTION

An allergic reaction is caused when the immune system attacks a foreign protein known as an allergen that has gained access to the body. Allergic reactions are characterized by a complex interaction of various inflammatory mediators but are generally triggered by an immunoglobulin E (IgE)-mediated response to a foreign protein, with the most common foreign protein being pollen. Exposure to these foreign proteins can lead to allergic sensitization, which is further characterized by the production of IgE against foreign proteins. The IgE coats the surface of mast cells present in the nasal mucosa. When the foreign proteins are introduced into the nasal passage, they may bind to the IgE on the mast cells leading to a release of several mediators including histamine, tryptase, chymase, kinins and heparin. Other mediators including leukotrienes and prostaglandin D2 are later released. The release of these various mediators leads to the symptoms of allergies or allergic rhinitis. The mediators can also lead to the presence of other inflammatory cells in the mucosa, such as neutrophils, eosinophils, lymphocytes, and macrophages which results in more inflammation and ongoing symptoms.

Rhinitis is defined as inflammation of the nasal membranes and is characterized by any combination of symptoms including sneezing, nasal congestion, nasal itching, and rhinorrhea. Allergic rhinitis is the most common cause of rhinitis and is commonly known as hay fever. Allergic rhinitis mimics a chronic cold and involves inflammation of the mucous membranes of the nose, eyes, Eustachian tubes, middle ear, sinuses and pharynx. Allergic rhinitis symptoms include sneezing, nasal congestion, headache, red eyes, tearing, earache, eye swelling, nasal itching, rhinorrhea, fatigue, and drowsiness. Post-nasal dripping of clear mucous frequently causes a cough. Eye itching, redness and tearing, known as “allergic conjunctivitis” is also frequently noted. Allergic rhinitis can be associated with other diseases such as sinusitis and asthma. While not life threatening by itself, allergic rhinitis can also be associated with several other complications such as otitis media, Eustachian tube dysfunction, nasal polyps and atopic dermatitis.

Allergic rhinitis implies hypersensitive response following exposure to allergens including pollens, animal danders, mold, and dust. Seasonal allergic rhinitis, which occurs at a specific time of year, is commonly triggered by exposure to pollens of grass, weeds, and trees; mold spores; animal danders; and dust. Perennial allergic rhinitis, which persists throughout the year, is commonly triggered by exposure to dust, animal dander, and molds. Sporadic allergic rhinitis is commonly triggered by infrequent exposure to animal dander, pollens, and molds. Occupational allergic rhinitis is another form which occurs due to exposure to specific allergens found in the workplace, such as animals, dust, or wood.

A general treatment of allergies is to avoid the allergens which trigger a response. Avoiding allergens which trigger a response is not practical due to the fact that triggering allergens may not be known and if known may be widespread and prevalent. Symptomatic treatments of allergies routinely used include antihistamines, decongestants, eye drops, cromolyn sodium, intranasal corticosteroids, topical decongestants, and glucocorticoids. However, symptomatic treatments generally do not alone treat all allergy symptoms experienced and commonly produce side effects such as drowsiness. For example, antihistamines do not treat all of the symptoms of allergies and commonly produce drowsiness, other side effects and sometimes CNS depression; decongestants only relieve congestion and not other allergy symptoms; and eye drops only relieve symptoms of allergies related to the eye. Allergy shots or immunotherapy, is also used to reduce sensitivity to specific allergens. However, allergy shots are time consuming, expensive, and not effective if many triggering allergens exist.

Tinospora cordifolia is a deciduous climbing shrub indigenous to the tropical Indian subcontinent. It has been used in Ayurvedic medicine for many years to maintain and strengthen the body's immune system, and for the management of diabetes, arthritis, fever, urinary tract infections, and some skin disorders. The use of this plant as an anti-inflammatory, and as an immuno-modulator for treatment of osteomyelitis, bronchitis, AIDS, flu, hepatitis, tuberculosis and diabetes has been studied but the use of Tinospora cordifolia in the treatment of allergies has not heretofore been evaluated.

A composition for use in a nutritional product, dietary supplement, food or pharmaceutical compound wherein such composition includes the plant Tinospora cordifolia or Tinospora cordifolia extract and is used to treat allergies is needed which is natural, practical, and not time consuming or expensive; alone provides treatment for all symptoms of allergies; does not produce drowsiness or other side effects; and improves quality of life. The present invention provides an effective treatment for all symptoms of allergies which is natural, practical, and not time consuming or expensive; improves quality of sleep; does not produce drowsiness or other side effects; and improves quality of life. There is also a need for a method of extracting and obtaining a composition for use in a nutritional product, dietary supplement, food or pharmaceutical compound wherein such composition includes the plant Tinospora cordifolia or Tinospora cordifolia extract and is used to treat allergies and alone provides treatment for all symptoms of allergies; is natural, practical, and not time consuming or expensive; and does not produce drowsiness or other side effects.

### SUMMARY OF THE INVENTION

The present invention relates to a composition for use in a nutritional product, dietary supplement, food or pharmaceutical composition wherein such composition includes the plant Tinospora cordifolia or Tinospora cordifolia extract and is used to treat allergies, including but not limited to seasonal, perennial, or sporadic allergic rhinitis. The present invention further relates to an extraction process for obtaining such composition.

A general object of this invention is to provide a natural, effective composition for use in a nutritional product, dietary supplement, food or pharmaceutical compound wherein such composition is used to treat allergies.

Another general object of this invention is to provide a natural, effective composition for use in a nutritional product, dietary supplement, food or pharmaceutical compound wherein such composition includes Tinospora cordifolia or Tinospora cordifolia extract and is used to treat allergic rhinitis.

Another general object of this invention is to provide a composition for use in a veterinary product wherein such composition includes Tinospora cordifolia or Tinospora cordifolia extract and is used to treat allergic rhinitis.

A further object of this invention is to provide a method of extracting and producing such composition.

More particularized objects of this invention are to treat all symptoms of allergies, to provide a treatment for allergies which does not produce drowsiness or other side effects, and to provide a treatment for allergies which is practical, and not time consuming or expensive.

Other beneficial uses of this invention are to provide a natural, effective composition for use in a nutritional product, dietary supplement, food or pharmaceutical compound wherein such composition includes Tinospora cordifolia or Tinospora cordifolia extract and is used to treat thermal burns, human immunodeficiency virus (HIV), sickle cell anemia, leprosy, and tuberculosis, and to strengthen immune system effects after receiving chemotherapy.

The present invention relates to a composition for use in a nutritional product, dietary supplement, food or pharmaceutical composition wherein such composition includes the plant Tinospora cordifolia or Tinospora cordifolia extract and is used to treat allergies, including but not limited to seasonal, perennial, or sporadic allergic rhinitis. The present invention further relates to an extraction process for obtaining such composition. The prior art has failed to provide an effective, natural, practical treatment for all symptoms of allergies which improves quality of sleep; does not produce drowsiness or other side effects; improves quality of life; and is not expensive or time consuming. The prior art has also failed to provide a method of extracting such composition.

### DETAILED DESCRIPTION OF THE INVENTION

The present invention relates to a composition for use in a nutritional product, dietary supplement, food or pharmaceutical composition wherein such composition includes the plant Tinospora cordifolia or Tinospora cordifolia extract and is used to treat allergies. The present invention further relates to an extraction process for obtaining such composition.

Tinospora cordifolia is a deciduous climbing shrub indigenous to the tropical Indian subcontinent, belonging to the family Menispermaceae. Common names for Tinospora cordifolia include Gulanshe Tinospara, Gulancha Tinospara, Tinospara, Giloya, and Guduchi. Tinospora cordifolia has been used in Ayurvedic medicine to maintain and strengthen the body's natural immune system by scavenging free radicals, and reducing adverse immune reactions that produce inflammation; to support liver function and improve digestion. It is purported to support the entire immune system by increasing the body's ability to fight infections through building up white blood cells and supporting inflammatory-modulating effects. Use of the present invention demonstrates promotion and enhancement of the immune system and a decrease in all the symptoms of allergies, including but not limited to an increase in total leukocyte count; a decrease in eosinophils and neutrophils; a decrease in the bronchospasm induced by 5% Histamine aerosol in guinea pigs; a decrease in capillary permeability in mice; and reduction in the number of disrupted mast cells in rats.

The composition of the present invention includes, without limitation, a Tinospora cordifolia extract in a therapeutically effective amount for the treatment of allergies. In one preferred embodiment, the composition is for the treatment of allergic rhinitis, including but not limited to seasonal, perennial, or sporadic allergic rhinitis.

In one preferred embodiment, the Tinospora cordifolia extract includes B-sitosterol. B-sitosterol, is a phyto-sterol, which is known for enhancing and supporting the immune system by balancing immune system response and modulating white blood cells.

The Tinospora cordifolia extract preferably includes an alkaloid component. In one preferred embodiment, the alkaloid component includes tinosporine or similarly therapeutic alkaloid. Tinosporine and alkaloids are known for enhancing and supporting the immune system.

The Tinospora cordifolia extract preferably includes a glycoside component. In one preferred embodiment, the glycoside component includes at least one of the following: cordifoliside A, cordifoliside B or similarly therapeutic combinations thereof. Cordifoliside A and cordifoliside B are known for enhancing and supporting the immune system.

In another preferred embodiment, the Tinospora cordifolia extract includes a diterpenoid component. In one preferred embodiment, the diterpenoid component includes tinosporaside or similarly therapeutic diterpenoid. Diterpenoids and tinosporaside are also known for enhancing and supporting the immune system.

Preferably the Tinospora cordifolia extract includes a polyphenol component. The Tinospora cordifolia extract preferably includes a polysaccharide component. The polyphenols and polysaccharides are also known for enhancing and supporting the immune system.

In one preferred embodiment, the Tinospora cordifolia extract includes B-sitosterol, an alkaloid component, a glycoside component, a diterpenoid component, a polyphenol component and a polysaccharide component the formulation including at least one or more of the following ingredients:
B-sitosterol;
Tinosporine;
Cordifoliside A;
Cordifoliside B;
Tinosporaside;
Other Diterpenoids;
Polyphenols; and
Polysaccharides.
   The individual components may be included in the composition in any of their pharmaceutically active forms.

The present invention further relates to a composition for use in a veterinary product wherein such composition includes the plant Tinospora cordifolia or Tinospora cordifolia extract and is used to treat allergies.

The composition of the present invention further includes, without limitation, a Tinospora cordifolia extract in a therapeutically effective amount for the treatment of at least one of the following conditions: thermal burns, human immunodeficiency virus (HIV), sickle cell anemia, leprosy, tuberculosis, and immune system deficiencies resulting from chemotherapy.

In one preferred embodiment, the composition is suitable for oral administration, although any suitable route of administration may be used in a therapeutically effective dosage of the composition. Other routes of administration include, for example, parenteral, intravenous, topical, or other like forms of administration. The preferable dosage form for oral administration includes but is not limited to tablets, capsules, gel caps, caplets or other suitable forms of oral administration. The preferable dosage form for other routes of administration include but are not limited to nasal spray or mist, solutions, liquids, suppositories or other suitable forms for administration other than oral administration.

### EXAMPLE

Use of the present invention wherein a 300 mg tablet of Tinospora cordifolia extract is administered three times daily demonstrates promotion and enhancement of the immune system and a decrease in all the symptoms of allergies, including but not limited to an increase in total leukocyte count and a decrease in eosinophils and neutrophils.

The present invention further relates to a method for extracting a composition for use in a nutritional product, dietary supplement, food or pharmaceutical composition wherein such composition includes the plant Tinospora cordifolia or Tinospora cordifolia extract and is used to treat allergies.

The method for extracting the composition for use in a nutritional product, dietary supplement, food or pharmaceutical composition wherein such composition includes the plant Tinospora cordifolia or Tinospora cordifolia extract and is used to treat allergies includes the steps of: obtaining and crushing raw Tinospora cordifolia to obtain a first Tinospora cordifolia extract; charging the first Tinospora cordifolia extract with water; refluxing the first Tinospora cordifolia extract and water; filtering the first Tinospora cordifolia extract to obtain a second Tinospora cordifolia extract; charging the second Tinospora cordifolia extract with water; refluxing the second Tinospora cordifolia extract and water; filtering the second Tinospora cordifolia extract to obtain a third Tinospora cordifolia extract; distilling the third Tinospora cordifolia extract to obtain a Tinospora cordifolia paste; and drying the Tinospora cordifolia paste to obtain the composition.

In one preferred embodiment, the raw Tinospora cordifolia is preferably obtained from dried stem and crushed in small pieces of various sizes. The amount of crushed raw Tinospora cordifolia is determined by the required amount of the composition and is preferably approximately 500 kg. The first and second Tinospora cordifolia extracts are preferably charged with approximately 1950-2050 L of deionized water. The first and second Tinospora cordifolia extracts and water are preferably refluxed for approximately 3 hours at a temperature of approximately 98-102° C. The filtering of the first and second Tinospora cordifolia extracts is preferably done through a press filter. The distillation of the third Tinospora cordifolia extract is preferably done on a rising film evaporator. The Tinospora cordifolia paste obtained from distilling the third extract preferably contains 35% solid Tinospora cordifolia extract.

In another preferred embodiment, the Tinospora cordifolia paste is dried in a vacuum dryer to obtain Tinospora cordifolia granules without an excipient. The vacuum drying of the paste is preferably done for approximately six (6) hours. The dried Tinospora cordifolia granules are preferably sifted through a mesh preferably of size 60. Preferably approximately 100 L of paste will yield approximately 30-40 kg of dried Tinospora cordifolia granules.

In a further preferred embodiment, the Tinospora cordifolia paste is spray dried to obtain Tinospora cordifolia powder. The spray dryer is preferably maintained at an inlet temperature of approximately 165° C. and an outlet temperature of approximately 100-102° C. The Tinospora cordifolia powder is preferably sifted through 100 mesh or other mesh size to obtain desired powder size.

In a further preferred embodiment, the Tinospora cordifolia paste is dried by fluid bed drying to obtain Tinospora cordifolia granules. The Tinospora cordifolia paste is mixed with an excipient, preferably gum acacia, starch, micro crystalline cellulose or other similar excipient. The Tinospora cordifolia paste and excipient are preferably put in a rapid mixer granulator for approximately ten minutes. Then the Tinospora cordifolia paste and excipient are preferably dried by fluid bed drying. The Tinospora cordifolia granules are preferably sifted through 40 or 60 mesh size depending upon desired size.

While in the foregoing specification this invention has been described in relation to certain preferred embodiments thereof, and many details have been set forth for the purpose of illustration, it will be apparent to those skilled in the art that the invention is susceptible to additional embodiments and that certain details described herein can be varied considerably without departing from the basic principles of the invention.

We claim:

1. A composition for use in a nutritional product, dietary supplement, food or pharmaceutical composition comprising Tinospora cordifolia extract for the treatment of allergies.

2. The composition of claim 1 wherein an allergy of the allergies comprises allergic rhinitis.

3. The composition of claim 1 wherein the Tinospora cordifolia extract comprises at least one of the following: B-sitosterol, an alkaloid component, a glycoside component, a diterpenoid component, a polyphenol component, and a polysaccharide component.

4. The composition of claim 3 wherein the alkaloid component comprises Tinosporine.

5. The composition of claim 3 wherein the glycoside component comprises at least one of the following: Cordifoliside A and Cordifoliside B.

6. The composition of claim 3 wherein the diterpenoid component comprises Tinosporaside.

7. The composition of claim 1 wherein the Tinospora cordifolia extract comprises at least one of the following: B-sitosterol, Tinosporine, Cordifoliside A, Cordifoliside B, Tinosporaside, a polyphenol and a polysaccharide.

8. The composition of claim 1 wherein the Tinospora cordifolia extract comprises a nasal spray form.

9. The composition of claim 1 for use in a veterinary product.

10. A method for extracting a composition for use in a nutritional product, dietary supplement, food or pharmaceutical composition comprising Tinospora cordifolia extract for the treatment of allergies which comprises:
obtaining and crushing raw Tinospora cordifolia to obtain a first Tinospora cordifolia extract;
charging the first Tinospora cordifolia extract with water;
refluxing the first Tinospora cordifolia extract and water;
filtering the first Tinospora cordifolia extract to obtain a second Tinospora cordifolia extract;
charging the second Tinospora cordifolia extract with water;
refluxing the second Tinospora cordifolia extract and water;
filtering the second Tinospora cordifolia extract to obtain a third Tinospora cordifolia extract;
distilling the third Tinospora cordifolia extract to obtain a Tinospora cordifolia paste; and
drying the Tinospora cordifolia paste to obtain the composition.

11. The method of claim 10 wherein the charging the first and second Tinospora cordifolia extracts with water comprises charging the first and second Tinospora cordifolia extracts with approximately 1950-2050 L of deionized water.

12. The method of claim 10 wherein the refluxing the first and second Tinospora cordifolia extracts with water comprises refluxing the first and second Tinospora cordifolia extracts for approximately 3 hours at a temperature of approximately 98-102° C.

13. The method of claim 10 wherein the Tinospora cordifolia paste comprises approximately 35% solid Tinospora cordifolia extract.

14. The method of claim 10 wherein the drying the Tinospora cordifolia paste comprises drying the Tinospora cordifolia paste in a vacuum dryer to obtain Tinospora cordifolia granules.

15. The method of claim 14 wherein the drying the Tinospora cordifolia paste in a vacuum dryer to obtain Tinospora cordifolia granules comprises drying the Tinospora cordifolia paste for approximately 6 hours.

16. The method of claim 14 wherein the drying the Tinospora cordifolia paste in a vacuum dryer to obtain Tinospora cordifolia granules comprises sifting the Tinospora cordifolia granules through a size 60 mesh.

17. The method of claim 10 wherein drying the Tinospora cordifolia paste comprises spray drying the Tinospora cordifolia paste to obtain a Tinospora cordifolia powder.

18. The method of claim 17 wherein spray drying the Tinospora cordifolia paste to obtain a Tinospora cordifolia powder comprises spray drying the Tinospora cordifolia paste at an inlet temperature of approximately 165° C. and an outlet temperature of approximately 100-102° C.

19. The method of claim 17 wherein spray drying the Tinospora cordifolia paste to obtain a Tinospora cordifolia powder comprises sifting the Tinospora cordifolia powder through a size 100 mesh.

20. The method of claim 10 wherein the drying the Tinospora cordifolia paste comprises drying the Tinospora cordifolia paste by fluid bed drying with an excipient to obtain Tinospora cordifolia granules.

21. The method of claim 20 wherein the excipient comprises at least one of the following: gum acacia, starch and micro crystalline cellulose.

22. The method of claim 20 wherein the drying the Tinospora cordifolia paste by fluid bed drying with an excipient to obtain Tinospora cordifolia granules comprises drying the Tinospora cordifolia paste for approximately 10 minutes.

23. The method of claim 20 wherein the drying the Tinospora cordifolia paste by fluid bed drying with an excipient to obtain Tinospora cordifolia granules comprises sifting the Tinospora cordifolia powder through a size 40 or 60 mesh.
